(19) United States
(12) Patent Application Publication
(10) Pub. No.: US 2007/0265483 A1
(43) Pub. Date: Nov. 15, 2007

(54) PROCESS FOR SELECTIVELY HYDROGENATING BUTADIENE IN AN C4 OLEFIN STREAM CONTAINING A CATALYST POISON WITH THE SIMULTANEOUS ISOMERIZATION OF 1-BUTENE TO 2-BUTENE

(76) Inventor: Paul Benjerman HIMELFARB, Houston, TX (US)

Correspondence Address: SHELL OIL COMPANY, P O BOX 2463, HOUSTON, TX, 772522463, US

(21) Appl. No.: 11/744,151

(22) Filed: May 3, 2007

Related U.S. Application Data
(60) Provisional application No. 60/746,536, filed on May 5, 2006.

(51) Int. Cl. C07C 7/167; C07C 5/25; C07C 2/56; C07C 5/08

(52) U.S. Cl. 585/664; 585/709; 585/259

(57) ABSTRACT

A process for selectively hydrogenating butadiene that is contained in a C4 stream having a concentration of a catalyst poison and C4 olefins, including 1-butene, and further the process provides for the substantial isomerization of the 1-butene to 2-butene of the C4 olefin stream, wherein the process comprises contacting, under suitable process conditions, the C4 stream with a poison tolerant catalyst comprising an alumina support material and partially sulfided nickel on the alumina support material at a concentration in the range of from 5 to 40 weight percent nickel, and yielding a product having a minimal butadiene concentration and a reduced concentration 1-butene.

This application claims the benefit of U.S. Provisional Application No. 60/746,536 filed May 5, 2006, the entire disclosure of which is hereby incorporated by reference.

This invention relates to a process for selectively hydrogenating butadiene contained in a hydrocarbon stream containing butylenes while simultaneously isomerizing the 1-butene to 2-butene. In one embodiment of the invention, the hydrocarbon stream further has a concentration of catalyst poisons and the process allows for the processing of such a hydrocarbon stream.

U.S. Pat. No. 3,485,887 discloses a process for the selective hydrogenation and simultaneous isomerization of C4 hydrocarbon mixtures that contain butadiene. The process that is taught by this patent uses a catalyst having a Group VIII metal as a hydrogenation component. The preferred Group VIII metal is palladium, and the amount of Group VIII metal supported on the substrate is in the range of from 0.01 to 1% by weight. The process provides for the isomerization of 1-butene to 2-butene while hydrogenating the butadiene contained in a C4 hydrocarbon mixture. It is noted that there is no specific mention of the use of a nickel based hydrogenation catalyst; and, especially, there is no mention of a nickel based hydrogenation catalyst containing a concentration of nickel that is above one weight percent. Furthermore, this patent does not address the processing of a C₄ hydrocarbon feedstock that contains a significant concentration of what are typically considered tₒ be poisons to noble metal catalysts.

U.S. Pat. No. 4,132,745 discloses a process for the simultaneous hydrogenation of butadiene and isomerization 1-butene to 2-butene of a process feed by use of a pretreated noble metal catalyst. The noble metal catalyst comprises a noble metal that is preferably palladium supported on a carrier in an amount in the range of from 0.01 to 2 weight percent. The pretreatment of the catalyst deactivates it and provides a pretreated catalyst that is less sensitive to catalyst poisons. The pretreated catalyst also provides for a lower 1-butene isomerization temperature and reduced activity for olefins saturation. The noble metal catalyst is pretreated by contacting it under suitable treatment conditions with a sulfur compound followed by treatment with hydrogen. The process of this patent only applies to noble metal catalysts. It is important to note that the patent recognizes the deactivation effect of sulfur on noble metal catalysts, but it advantageously utilizes this effect to purposely make the catalyst of the process less active. While the patent mentions that the sensitivity of its treated catalyst to poisons is reduced as compared with the unpretreated noble metal catalysts, it only mentions butadiene and sulfur as being poisons. There is no mention of the use of nickel-based catalysts.

The invention of U.S. Pat. No. 4,260,840 concerns the selective hydrogenation of butadiene contained in a process stream containing 1-butene with a minimization of the isomerization of 1-butene to 2-butene. The catalyst is an alumina supported palladium catalyst having a palladium content of from 0.01 to 1 wt %. There is no mention, however, of the use of nickel-based catalysts. It is specifically recognized that the process of the '840 patent is intended to minimize rather than maximize the amount of 1-butene isomerization that occurs.

U.S. Pat. No. 6,686,510 discloses a multi-step process with one of the steps involving the selective hydrogenation of butadiene that is contained in a feed stream of 1-butene with the simultaneous isomerization of the 1-butene to 2-butene. The catalyst used for this step includes a group 10 metal, i.e., Ni, Pd, or Pt, deposited on a substrate. An advantageous catalyst is one that consists of palladium deposited on alumina and sulfur. The palladium content is present in the range of from 0.01 to 5% by weight. The catalyst may further be pretreated with sulfur. There is no mention of a nickel-based hydrogenation catalyst containing a concentration of nickel that is above five weight percent nor is the process of the '510 patent directed to the processing of a feed stream that contains a significant concentration of what are typically considered to be poisons to selective hydrogenation metal catalysts.

It is desirable to have a process that is highly selective in the hydrogenation of butadiene contained in a hydrocarbon feed stream that comprises butenes while simultaneously providing for the significant conversion of 1-butene that is contained in the hydrocarbon feed stream to 2-butene.

It is further desirable to have a process that is capable of handling the processing of a butene-containing hydrocarbon feed stream having a significant concentration of compounds that are traditionally considered to be catalytic poisons without a significant rate of deactivation of the catalyst of the process and to also provide for the simultaneous butene isomerization and butadiene hydrogenation.

Accordingly, a process is provided for selectively hydrogenating butadiene that is contained in a C4 feed stream having a 1-butene concentration while simultaneously providing for the isomerization of 1-butene to 2-butene of said C4 feed stream, wherein said process comprises: contacting, under suitable butadiene hydrogenation and butene isomerization process conditions, said C4 stream with a poison tolerant catalyst; and yielding a product having a minimal butadiene concentration and a reduced 1-butene concentration that is less than said 1-butene concentration.

The invention relates to a process for selectively hydrogenating butadiene contained in a hydrocarbon stream that further contains at least one butylene isomer (1-butene, cis and trans 2-butene, and isobutene) and butadiene. One of the advantages of the inventive process is that it is capable of selectively hydrogenating butadiene while simultaneously isomerizing butylenes of a C4 feed stream that also has a concentration of a catalyst poison but without causing a significant rate of loss in the activity of the catalyst of the process. The inventive process, thus, provides for the selective hydrogenation of butadiene that is contained in a hydrocarbon stream having a butene concentration without a significant amount of saturation of the butene contained in the hydrocarbon stream.

The hydrocarbon feed stream of the inventive process may be from any source that provides for a hydrocarbon mixture comprising at least one unsaturated hydrocarbon, such as, an olefin, having 4 carbon atoms. There are, in fact, a number of typical sources of such hydrocarbon mixtures. One possible source is from a steam cracking process for pyrolytically cracking hydrocarbons to produce ethylene and propylene. Steam cracking processes generally also yield a heavy hydrocarbon product from which can be separated a C4 hydrocarbon fraction that comprises at least one butene compound. Typically, such a C4 hydrocarbon fraction contains a significant concentration of butadiene as well as a significant concentration of at least one butene compound.

Another possible source of the hydrocarbon mixture is that which is yielded from the catalytic cracking of a heavy hydrocarbon, such as gas oil. The C4 hydrocarbon fraction resulting from the catalytic cracking of a heavy hydrocarbon typically contains a concentration of butadiene as well as including a significant concentration of one or more butene compounds. Such a C4 hydrocarbon fraction is often used as a feedstock for an alkylation process that provides for the alkylation of the olefin compounds with an isoparaffin compound to yield an alkylate product that is particularly suitable as a high octane motor gasoline blending component.

Thus, the C4 feed stream of the inventive process comprises at least one butene compound and butadiene. The at least one butene compound is one selected from the group of olefins consisting of 1-butene, trans-2-butene, cis-2-butene, and isobutylene. When the C4 feed stream is to be used as a feedstock for an hydrofluoric acid alkylation process, it is desirable for the olefin component to be 2-butene rather than 1-butene; because, it provides for a higher octane alkylate product. And, indeed, one of the advantages of the invention is that it provides for the high conversion isomerization of the less desirable 1-butene to the more desirable 2-butene while simultaneously hydrogenating the butadiene with a minimal amount of olefin saturation.

A typical C4 feed stream may comprise a concentration of 1-butene in the range of from 3 to 30 mole percent of the C4 feed stream, more typically, from 4 to 20 mole %, and, most typically, from 5 to 15 mole %. The C4 feed stream may further comprise a concentration of 2-butene (including both of the cis and trans diastereomers) in the range of from 8 to 28 mole %, more typically, from 10 to 26 mole %, and, most typically, from 12 to 24 mole %. While not desired, due to the sources of the C4 feed stream, it may have a butadiene concentration in the range of from 0.1 to 5 mole %, more typically, from 0.2 to 3 mole %, and, most typically, from 0.3 to 2 mole %, with the mole % being based on the total moles of hydrocarbon in the C4 feed stream. The molar ratio of 2-butene to 1-butene in the C4 feed stream of the inventive process is generally in the range of from 0.5:1 to 3:1, and, more typically, from 1:1 to 2:1.

Another advantage of the inventive process is its capability of processing a C4 feed stream that comprises a concentration of a catalyst poison without an uneconomically high rate of catalyst deactivation caused by the presence of such catalyst poison contained in the C4 feed stream. Thus, the C4 feed stream of the inventive process may contain a concentration of a catalyst poison, such as, compounds selected from the group consisting of arsenic compounds, chlorine compounds, sulfur compounds, and metal-containing compounds.

The concentration range of the catalyst poison contained in the C4 feed stream will vary depending upon the particular poison or poisons that are present and the source of the C4 feed stream, but the summation of all the poisons contained in the C4 feed stream of the inventive process generally will exceed 0.01 parts per million by weight (ppmw), but, more typically, the poison concentration can exceed 0.02 ppmw, and most typically, it exceeds 0.03 ppmw. The measurement of the poison concentration is based on the weight of the element itself (i.e., As, Cl, S, and Metal) of the poison compound rather than on the weight of the compound that contains the particular element. For the aforementioned poison concentration ranges, if for example the C4 feed stream contains an organic arsenic compound, an organic chlorine compound, an organic sulfur compound and an organic metal compound, wherein each of which is a poison to the catalyst, the determination of the concentration of poisons contained in the C4 feed stream is made by summing the elemental weights of arsenic, chlorine, sulfur, and metal and finding the ppmw of such elements that is present in the C4 feed stream. If only one poison such as an organic arsenic compound is present in the C4 feed stream, then the concentration of the catalyst poison is determined by the ppm weight of elemental arsenic that is present in the C4 feed stream.

Arsenic compounds considered to be catalyst poisons includes elemental arsenic and any other arsenic containing compound including organic arsenic compounds, the hydrides of arsenic, such as arsine (AsH₃), the oxides of arsenic, the halides of arsenic, and the sulfides of arsenic. The more typical arsenic compound contained in the C4 feed stream is an organic arsenic compound and is present therein in an amount exceeding 1 part per billion by weight (ppb) and, normally, is in the range of from 1 ppbw to 1000 ppbw. More typically, the arsenic compound is present in an amount in the range of from 5 ppbw to 500 ppbw, and most typically, from 10 ppbw to 100 ppbw.

The inventive process includes contacting of the C4 feed stream under suitable butadiene hydrogenation and butene isomerization process conditions with a poison tolerant selective hydrogenation and isomerization catalyst (poison tolerant catalyst) and yielding a product having a reduced butadiene concentration that is less than the butadiene concentration in the C4 feed stream and a reduced 1-butene concentration that is less than the 1-butene concentration of the C4 feed stream.

The composition of the poison tolerant catalyst is particularly important to the successful performance of the inventive process. It has been discovered that certain nickel-based catalyst compositions can provide for the simultaneous selective butadiene hydrogenation and butene isomerization. This is particularly unexpected; since, it has generally been thought that nickel-based catalysts do not have particularly high isomerization catalytic activity. The poison tolerant catalyst of the inventive process, therefore, includes certain nickel-based catalyst compositions that provide for the desired simultaneous, or dual, isomerization and hydrogenation while also being tolerant to catalyst poisons.

There are two important properties that are required of the nickel-based catalyst for it to be suitable for use as the poison tolerant catalyst of the inventive process. The nickel-based catalyst should have an appropriately high nickel content, and its activation should be controlled so as to provide the appropriate amount of sulfiding and activation to provide the desired ratio of sulfided nickel to nickel metal in the activated nickel-based catalyst (hereafter also referred to as partially sulfided nickel-based catalyst).

The nickel-based catalyst, in general, comprises a nickel catalytic component and an inorganic oxide material, which serves as either a support material or a binder material, or both. The nickel-based catalyst may be selected from the group of nickel-based catalysts consisting of a supported nickel catalyst made by the impregnation of an inorganic oxide support and a bulk nickel catalyst made by the coprecipitation of the various components of the bulk nickel catalyst. As already noted, the nickel content of the nickel-based catalyst of the invention is important and should be significantly high so as to provide a nickel-based catalyst having the desired properties.

While not wanting to be bound to any particular theory, it is, however, believed that a high nickel content is important to the inventive process in that it provides significantly more surface area than do noble metal catalysts thereby allowing for the adsorption of greater amounts of poisons. Thus, the nickel component of the supported nickel catalyst can be present therein in an amount that effectively provides the required catalytic surface area for use in the inventive process and can be in the range of from 5 weight percent to 40 weight percent, with the weight percent being based on the total weight of the nickel-based catalyst and the nickel component as elemental nickel. A preferred nickel content is in the range of from 8 wt. % to 30 wt. %, and, most preferred, from 10 wt. % to 20 wt. %. The inorganic oxide support is present in the supported nickel catalyst in an amount in the range of from 60 to 95 weight percent of the total weight of the supported nickel catalyst.

The bulk nickel catalysts are prepared by the coprecipitation of the components that make up the bulk nickel catalyst, which comprises a catalytic nickel component and an inorganic oxide component such as silica. The bulk nickel catalyst will, in general, contain a high amount of nickel as compared to typical supported nickel catalysts. The nickel content of the bulk nickel catalyst can be in the range of from 20 wt. % to 80 wt. %, with the weight percent being based on the total weight of the bulk nickel catalyst and the nickel component as elemental nickel. A preferred nickel content in the bulk nickel catalyst is in the range of from 25 wt. % to 70 wt. %, and, most preferred, from 30 wt. % to 60 wt. %. The inorganic oxide content of the bulk nickel catalyst may be in the range of from 20 wt. % to 80 wt. % based on the total weight of the bulk nickel catalyst.

The nickel-based catalysts of the invention may further include other components, including catalytic metals, provided that such other components do not significantly inhibit the dual hydrogenation and isomerization reactions or otherwise materially affect the catalytic performance of the nickel-based catalyst in the practice of the inventive process.

The inorganic oxide support material for the supported nickel catalyst or the inorganic oxide component of the bulk nickel catalyst may be selected from the group of refractory oxides consisting of alumina, silica, silica-alumina, titania, zirconia, and any combination of one or more thereof. Alumina, silica and mixtures thereof are particularly preferred inorganic oxide materials.

The other property of the nickel-based catalyst noted as being important to the invention is the requirement that the nickel component be partially sulfided such that the activated nickel-based catalyst comprises the appropriate ratio of sulfided nickel to nickel metal. This ratio is important to providing a poison tolerant catalyst having the correct amount of olefins saturation activity and selectivity. It is recognized that when the nickel is present in the nickel-based catalyst in a metallic form, it is highly active toward the saturation of olefins, but the metallic nickel does not provide for the required selectivity toward the saturation of diolefins as opposed to the saturation of monoolefins. On the other hand, when the nickel is present in the form of nickel sulfide, its olefins saturation activity is much reduced below that of nickel metal, but it is much more selective than is nickel metal toward the saturation of diolefins as opposed to the saturation of monoolefins.

The nickel-based catalyst of the invention is, thus, a partially sulfided nickel-based catalyst. It is generally desirable for upwardly to 90 weight percent of the nickel contained in the partially sulfided nickel-based catalyst to be present therein in the form of nickel sulfide and for upwardly to 90 weight percent of the nickel contained in the partially sulfided nickel-based catalyst to be present therein in the form of nickel metal. It is preferred for from 8 to 80 weight percent of the nickel of the partially sulfided nickel-based catalyst to be in the form of nickel sulfide and from 20 to 92 weight percent of the nickel of the partially sulfided nickel-based catalyst to be in the form of nickel metal. More preferred is for from 9 to 50 weight percent of the nickel in the partially sulfided nickel-based catalyst to be in the form of nickel sulfide and from 50 to 91 weight percent of the nickel to be in the form of nickel metal. And, most preferred, is for from 10 to 25 weight percent of the nickel of the partially sulfided nickel-based catalyst to be in the form of nickel sulfide and from 75 to 90 weight percent of the nickel to be the form of nickel metal.

Any of the methods know to those skilled in the art may be used to provide the partially sulfided nickel-based catalyst and, thus, the method is not necessarily critical; provided, that, it allows for the partial sulfiding of a nickel-based catalyst that is in an oxidic form to give the partially sulfided nickel-based catalyst having the desired relative amounts of sulfided nickel and nickel metal. In general, a fresh nickel-based catalyst in which the nickel component is present predominantly in the oxidic form, or a fresh nickel-based catalyst that has been reduced by the treatment with hydrogen, is treated with a sulfur compound under suitable sulfiding conditions. The sulfided nickel-based catalyst, if not already reduced, is further treated with hydrogen under suitable reducing conditions to thereby provide the partially sulfided nickel-based catalyst having the properties and compositions as detailed elsewhere herein.

To sulfide the fresh nickel-based catalyst, a sulfur compound is contacted with the fresh nickel-based catalyst at a treatment temperature in the range of from 100° C. to 500° C. The sulfur compound may be applied alone or diluted in a gas such as nitrogen, methane, argon, or hydrogen. It is also possible to use a sulfur compound that is in the liquid phase either in neat form or as a component of a hydrocarbon solution. Any suitable sulfur compound may be used to partially sulfide the nickel-based catalyst as long as it provides for the desired amount of sulfiding of the nickel component of the nickel-based catalyst. Possible suitable sulfur compounds include elemental sulfur, certain inorganic sulfur compounds such as H₂S and various other organic sulfur compounds.

The nickel-based catalyst is treated with hydrogen so as to reduce at least a portion of its nickel component that is in the oxidic form to nickel metal. This reduction step is carried out by contacting the nickel-based catalyst with high purity hydrogen at a treatment temperature in the range of from 300° C. to 500° C. The high purity hydrogen is a gas stream having a hydrogen content in the range of from 60 to 100 mole percent hydrogen. Other components of the high purity hydrogen may include sulfur compounds (for sulfiding), hydrocarbons and inert gases.

The partially sulfided nickel-based catalyst may have a BET surface area in the range of from 40 m²/g to 300 m²/g, preferably from 80 m²/g to 250 m²/g. The pore volume of the partially sulfided nickel-based catalyst may be in the range of from 0.3 ml/g to 0.9 ml/g, and, preferably, from 0.4 ml/g to 0.8 ml/g.

The contacting step of the invention may be carried out in the gas phase, or the liquid phase, or a mixed phase. Generally, any type of reactor system known to those skilled in the art may be used. One example of such a reactor system is a reactor vessel filled with a bed of the poison tolerant catalyst into which is introduced the C4 feed stream along with hydrogen which are contacted with the poison tolerant catalyst of the invention under selective hydrogenation and isomerization process conditions. Due to its use of the poison tolerant catalyst, the inventive process is able to treat C4 feed streams that have significant concentration levels of catalyst poisons and still maintain acceptable cycle lengths.

It is significant that the inventive process can provide for the conversion of at least 40 mole percent of the 1-butene component of the C4 feed stream to 2-butene. But, preferably, at least 60 mole percent of the 1-butene component of the C4 feed stream is converted to 2-butene, and, most preferably, at least 80 mole percent of the 1-butene component of the C4 feed stream is converted to 2-butene. The result from the significantly high conversion of the 1-butene is that the yielded product of the inventive process can comprise 2-butene and 1-butene in such amounts that the molar ratio of 2-butene to 1-butene in the product is at least 6:1, but, preferably the molar ratio of 2-butene to 1-butene in the product exceeds 8:1, and, more preferably, the molar ratio exceeds 12:1.

It is further noted that the inventive process unexpectedly provides for a significantly high amount of isomerization conversion of the 1-butene of the C4 feed stream to 2-butene with the application of a nickel-based catalyst. This family of catalysts has previously been considered to not have much isomerization activity as compared to noble metal catalysts. But, with the instant invention, the use of the poison tolerant catalyst can provide for isomerization activity that exceeds what is typically observed with the use of noble metal catalysts.

Another significant feature of the inventive process is that it can provide for the selective hydrogenation removal of butadiene contained in the C4 feed stream. What is meant by selective hydrogenation removal of butadiene is that a small amount of monoolefin saturation occurs but with a significant removal of the butadiene by hydrogenation. The inventive process can provide for greater than 97.5 percent removal of the butadiene contained in the C4 feed stream with less than 2 weight percent, or even less than 1 weight percent, or less than 0.5 weight percent, saturation of the monoolefins contained in the C4 feed stream.

It is preferred that more than 98 weight percent of the butadiene of the C4 feed stream to be removed therefrom, and, most preferably, more than 99 weight percent of the butadiene is to be removed from the C4 feed stream. Thus, the yielded product of the inventive process can further comprise a minimal butadiene concentration of less than 0.1 mole percent (1000 ppmm). It is preferred, however, for the concentration of butadiene in the yielded product of the inventive process to be less than 0.05 mole percent (500 ppmm), and, more preferably, less than 0.01 mole percent (100 ppmw).

Because the contacting step may be carried out under liquid phase or gas phase conditions there can be a reasonably broad range of suitable selective hydrogenation and isomerization process conditions at which the C4 feed stream is contacted with the sulfur tolerant catalyst. The reaction temperature as measured at the inlet of the reactor, i.e., the reactor inlet temperature, is broadly in the range of from 50° C. to 250° C., but preferably, the reaction temperature is in the range of from 70° C. to 200° C.

Concerning the reaction pressure, a desirable pressure varies significantly depending upon whether the process is operated as a liquid phase or gas phase or mixed phase process. Therefore, the reaction pressure can be in the broad range of from 5 bar (72 psia) upwardly to about 45 bar (650 psia). For liquid phase operation of the inventive process, the reaction pressure can be in the range of from 15 bar (217 psia) to 45 bar (650 psia). For gas phase operation of the inventive process, the reaction pressure can be in the range of from 5 bar to 15 bar.

The space velocity at which the inventive process is operated also can vary significantly depending upon whether the process is operated as a liquid phase or gas phase or mixed phase process. For gas phase operation, the gaseous hourly space velocity (GHSV) can be in the range of from 5 hr⁻¹ to 1000 hr⁻¹, preferably, in the range of from 15 hr⁻¹ to 500 hr⁻¹, and, most preferably, from 25 hr⁻¹ to 250 hr⁻¹. For liquid phase operation, the liquid hourly space velocity (LHSV) can be in the range of from 0.1 hr⁻¹ to 30 hr⁻¹, preferably, from 0.5 hr⁻¹ to 20 hr⁻¹, and, most preferably, from 1 hr⁻¹ to 10 hr⁻¹.

The amount of hydrogen contacted along with the C4 feed stream with the poison tolerant catalyst may be such as to provide a molar ratio of hydrogen to hydrocarbon in the range of from 0.005:1 to 0.3:1 moles H₂ per moles of hydrocarbon in the C4 feed stream. The preferred molar ratio of hydrogen to hydrocarbon is in the range of from 0.008:1 to 0.2:1, and, most preferred, from 0.01:1 to 0.1:1. The hydrogen consumption will approximate that which is expected from a stoichiometric standpoint, which is basically the amount of hydrogen required to hydrogenate the butadiene contained in the C4 feed stream and the amount of hydrogen consumed as a result of the saturation of other olefins and excess to promote isomerization.

A further application of the process for selectively hydrogenating butadiene that is contained in a C4 feed stream includes its coupling with an alkylation process for the alkylation of butene by isobutane to yield an alkylate product. In this embodiment, the product yielded from the selective hydrogenation and isomerization process is introduced into an alkylation reactor wherein it is contacted along with isobutane with an alkylation catalyst and under alkylation reaction conditions to thereby yield an alkylate product. By combining the inventive selective hydrogenation and isomerization process with the alkylation process, a high quality alkylate product, as reflected by its research octane number (RON), is produced. Specifically, the coupling of the two process steps can provide for an enhancement in the alkylate product RON of more than 2 research octane numbers, and even more than 3 research octane numbers over and above the RON of the alkylate that is yielded by processing or alkylating the C4 feed stream that has not been treated by the inventive selective hydrogenation and isomerization process.

That which is claimed is:

1. A process for selectively hydrogenating butadiene that is contained in a C4 feed stream having a 1-butene concentration while simultaneously providing for the isomerization of 1-butene to 2-butene of said C4 feed stream, wherein said process comprises:
contacting, under suitable butadiene hydrogenation and butene isomerization process conditions, said C4 stream with a poison tolerant catalyst; and
yielding a product having a minimal butadiene concentration and a reduced 1-butene concentration that is less than said 1-butene concentration.

2. A process as recited in claim 1, wherein said poison tolerant catalyst is a nickel based catalyst comprising an alumina support material and a nickel component present in said nickel based catalyst in an amount in the range of from 5 wt % to 40 wt %, wherein the wt % is based on the nickel in the elemental form and the total weight of said nickel based catalyst.

3. A process as recited in claim 2, wherein said nickel component comprises partially sulfided nickel.

4. A process as recited in claim 3, wherein said C4 feed stream contains butadiene at a butadiene concentration in the range of from 0.1 mole percent to 5 mole percent of the total moles of hydrocarbon of said C4 feed stream.

5. A process as recited in claim 4, wherein said C4 feed stream includes said 1-butene concentration in the range of from 3 mole % to 30 mole % of said C4 feed stream.

6. A process as recited in claim 5, wherein said C4 feed stream comprises a feed molar ratio of 2-butene to 1-butene is in the range of from 0.5:1 to 3:1 and a catalyst poison concentration of a catalyst poison exceeding 0.01 ppmw.

7. A process as recited in claim 6, wherein said catalyst poison includes a compound selected from the group consisting of arsenic compounds, chlorine compounds, sulfur compounds and metal-containing compounds.

8. A process as recited in claim 7, wherein said catalyst poison is an arsenic compound present in said C4 feed stream at said catalyst poison concentration in the range of from 1 ppbw to 1000 ppbw.

9. A process as recited in claim 8, wherein said product comprises said minimal butadiene concentration of less than 1000 ppmm and product molar ratio of 2-butene to 1-butene of at least 6:1.

10. A process as recited in claim 9, further comprising: passing said product as an alkylation feed to an alkylation system for alkylating monoolefins with an isoparaffin to yield an alkylate product.
